(19) United States
(12) Patent Application Publication
(10) Pub. No.: US 2023/0158004 A1
(43) Pub. Date: May 25, 2023

(54) ENTERIC-COATED PREPARATION COMPRISING PIRFENIDONE HAVING IMPROVED SAFETY AND STABILITY, AND METHOD FOR PREPARING SAME

(71) Applicant: YUNGJIN PHARM. CO., LTD., Seoul (KR)

(72) Inventors: Yangno YOON, Seongnam-si (KR); Byoung Ki KIM, Yongin-si (KR); Seung Yun BACK, Osan-si (KR); Young Hun KIM, Suwon-si (KR)

(21) Appl. No.: 17/919,855

(22) PCT Filed: Apr. 22, 2021
(86) PCT No.: PCT/KR2021/005123

§ 371 Date: Oct. 19, 2022

(30) Foreign Application Priority Data
Apr. 22, 2020 (KR) 10-2020-0048924

(51) Int. Cl. A61K 31/4418; A61K 9/28

(52) U.S. Cl. CPC A61K 31/4418; A61K 9/2866; A61K 9/2846; A61K 9/284; A61K 9/2886

(57) ABSTRACT

The present invention relates to a pirfenidone formulation having improved safety and a method for producing same, wherein the pirfenidone formulation comprises pirfenidone as an active component, has two or more mutually different coatings selected from the group consisting of a coating containing a water-soluble polymer and a coating containing an enteric polymer, thereby specifically releasing pirfenidone in the small intestine, and has unique pharmacokinetic properties to reduce variability when pirfenidone is absorbed through the gastrointestinal tract, and reduce gastrointestinal side effects without affecting bioavailability of the active component, and therefore can remarkably improve low medication compliance.

### TECHNICAL FIELD

The present invention relates to an enteric-coated formulation having improved safety and stability for human body, which includes pirfenidone as an active component, specifically releases pirfenidone in the small intestine and has unique pharmacokinetic properties, and a method for producing the same.

### BACKGROUND ART

Pirfenidone is an antifibrotic drug that is useful for the treatment of idiopathic pulmonary fibrosis, and has a production-regulating action on various cytokines and growth factors, for example, indicates inhibition of production of inflammatory cytokines (TNF-α, IL-1, IL-6) and enhancement of production of anti-inflammatory cytokines (IL-10), indicates suppression of the decrease in IFN-γ level leading to improvement of the Th2-type deflection (correction of Th1 Th2 balance), and indicates inhibition of production of growth factors (TGF-β, b-FGF, PDGF) involved in fibrosis formation. In addition, pirfenidone also has a fibroblast growth inhibitory action and collagen production inhibitory action, and exhibits an antifibrotic action based on these complex actions. In regard to the various effects of pirfenidone, therapeutic effects on pulmonary fibrosis, arteriosclerotic lesions, etc. are described in Japanese Unexamined Patent Publication No. H02-215719, its utility for the treatment of inflammation of the respiratory tract or skin are described in U.S. Pat. Nos. 3,974,281, 4,042,699 and 4,052,509, and the effect of inhibiting the synthesis and release of TNF-α is described in Japanese Unexamined Patent Publication No. H11-512699.

Meanwhile, idiopathic pulmonary fibrosis is a disease in which fibrosis progresses in the alveolar wall due to an unconfirmed cause, which gradually decreases lung function, such as lung activity, and eventually leads to respiratory distress. This is also a disease with a poor prognosis sufficient for approximately 50% of patients to reach death within 5 years, and is defined as a rare intractable disease in South Korea.

It has been reported that in a global clinical trial involving healthy adults and patients with idiopathic pulmonary fibrosis (a total 1,650 people), such pirfenidone caused nausea (32.4%), rash (26.2%), diarrhea (18.8%), indigestion (16.1%), anorexia (11.4%), headache (10.1%), and photosensitivity (9.3%).

The initial dose of pirfenidone is orally administered 1 tablet after meal, three times a day. The dose may be increased by 1 tablet at an interval of 2 weeks depending on the patient's response and tolerability. Since the dose can be increased up to 3 times a day, 3 tablets at a time (600 mg at a time, 1800 mg per day), it is cumbersome and inconvenient to take, and the rate of side effects is high. Therefore, development of a safe formulation that eliminates these problems and improves medication compliance is required. In addition, along with the development of a safe pirfenidone formulation, development of pirfenidone and a formulation containing the same (e.g., tablet) that minimize the generation of related compounds and stably maintains pirfenidone until administration after manufacture has also been requested. In this way, if the safety of the formulation in the human body and the stability of the drug decrease, the expected effect of tablet development will decrease, and the desired degree of therapeutic or preventive effect will not be obtained. Therefore, there is a need to develop pirfenidone-containing formulations that have secured safety and stability.

### DETAILED DESCRIPTION OF THE INVENTION

### Technical Problem

An object of the present invention is to provide an enteric-coated formulation having improved safety and stability, comprising pirfenidone, and a method for producing the same, more specifically, to improve the safety and stability of pirfenidone enteric-coated formulations which may be lowered by an enteric coating base, and a method for manufacturing the same.

### Technical Solution

In order to achieve the above object, according to one embodiment of the present invention, there is provided a formulation comprising pirfenidone as an active component and having improved safety and drug stability for human body.

In one embodiment, the formulation according to the present invention may be a formulation for oral administration.

In one embodiment, the formulation according to the present invention may be an enteric-coated formulation, more specifically, an enteric-coated tablet.

In one embodiment, the formulation according to the present invention may be a formulation for preventing or treating idiopathic pulmonary fibrosis.

In one embodiment, the formulation according to the present invention may be a formulation for oral administration, characterized in that pirfenidone as an active component exhibits a specific pharmacokinetic pattern. Specifically, it may be a formulation for preventing or treating idiopathic pulmonary fibrosis, characterized in that the time to reach the maximum blood concentration (Tₘₐₓ) at the time of administration is 1.2 to 1.4 hours after administration.

In one embodiment, the formulation according to the present invention may be a formulation for oral administration, in which in regard to the stability of the drug, the stability is improved in the stability test under severe conditions (60±2° C.) for 1 month and accelerated conditions (40±2° C., 75±5% RH) for 6 months, that is, related compound A is 0.1% or less, related compound B is 0.1% or less, individual unknown related compounds are 0.05% or less, and total related compounds are 0.3% or less.

Preferably, the formulation according to the present invention may include a core containing pirfenidone and a pharmaceutically acceptable additive; and two or more mutually different coatings located outside the core. More preferably, the two or more mutually different coatings may be two or more types selected from the group consisting of a coating containing a water-soluble or insoluble polymer and a coating containing an enteric polymer.

According to another embodiment of the present invention, there is provided a method for producing the formulation for preventing or treating idiopathic pulmonary fibrosis comprising pirfenidone having improved safety and stability, the method comprising a step of preparing a core using pirfenidone and a pharmaceutically acceptable additive; and a step of forming two or more types of coatings using two or more types of polymers on the prepared core. Preferably, the two or more mutually different coatings can be made sequentially, or can be performed by forming through the step of performing a primary coating using a water-soluble or insoluble polymer and the step of performing a secondary coating using an enteric polymer.

### Advantageous Effects

The pirfenidone enteric-coated formulation according to the present invention uses a methacrylic acid copolymer as an enteric coating base, whereby it not only specifically releases pirfenidone in the small intestine and has unique pharmacokinetic properties while excellently retaining the stability of pirfenidone, but also can alleviate or reduce the occurrence of gastrointestinal side effects caused by administration of pirfenidone without affecting the bioavailability of pirfenidone, and can significantly improve patient medication compliance, which is very useful in the development of oral formulations of pirfenidone.

### BRIEF DESCRIPTION OF THE DRAWINGS

FIG. 1 is a graph showing the results of elution test of the pirfenidone formulations according to Examples 1 to 8 and Comparative Example 1 at pH 1.2 as cumulative elution rates over time for 0 to 120 minutes.

FIG. 2 is a graph showing the results of elution test of the pirfenidone formulations according to Examples 1 to 8 and Comparative Example 1 at pH 6.8 as cumulative elution rates over time for 0 to 120 minutes.

FIG. 3 is a graph showing the results of a pharmacokinetic analysis test, in which the pirfenidone formulations according to Examples 1 and 8 and Comparative Example 1 are administered to beagle dogs, as the blood concentration of pirfenidone over time.

FIG. 4 is a graph showing the total related compounds among the stability test results of the pirfenidone formulations of Examples 9 to 11 and Examples 1 and 3 under severe conditions (60±2° C.).

FIG. 5 is a graph showing the total related compounds among the stability test results of the pirfenidone formulations of Examples 9 to 11 and Examples 1 and 3 under accelerated conditions (40±2° C., 75±5% RH).

### DETAILED DESCRIPTION OF THE EMBODIMENTS

Hereinafter, the present invention will be described in more detail.

The formulation according to the present invention comprises pirfenidone as an active component, and thus is a formulation useful for preventing or treating idiopathic pulmonary fibrosis. Specifically, pirfenidone contained as an active component (main component) in the formulation for oral administration according to the present invention is a drug composed of small molecules, and its chemical name is 5-methyl-1-phenyl-2-(1H)-pyridone. It is a non-peptide synthetic molecule with a molecular weight of 185.23 Daltons. Its chemical formula is C₁₂H₁₁NO, and its structure is the same as shown in the following Chemical Formula 1.

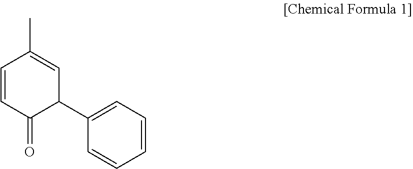

Currently, pirfenidone is clinically evaluated as a broad-spectrum antifibrotic drug. Pirfenidone is known to possess antifibrotic and anti-inflammatory pharmacological activity, which is reflected in its activity of reducing the expression of TGF-01, TNF-α, PDGF, and most importantly, the expression of different types of collagens.

As used herein, the term “time to reach maximum blood concentration (Tₘₐₓ)” means the time to reach the highest concentration (Cₘₐₓ) in blood after administration of a drug. The formulation according to the present invention is characterized in that the time to reach the maximum blood concentration (_{Tmax}) of pirfenidone contained as the active component is 1.2 hours, 1.4 hours or more, 1.6 hours or more, 2 hours or more, and 2 hours to 4 hours after administration. In addition, the formulation for preventing or treating idiopathic pulmonary fibrosis according to the present invention is characterized in that it does not substantially release pirfenidone within 1 hour after administration. When it does not substantially release pirfenidone within 1 hour after administration in this way, the cumulative blood concentration in the body within 1 hour after administration is 0 to 50%, 0 to 30%, preferably 0 to 10%, more preferably 0 to 5% of the maximum blood concentration (Cₘₐₓ).

In a preferred embodiment, the formulation for preventing or treating idiopathic pulmonary fibrosis according to the present invention is characterized in that the ratio of the area under the blood concentration curve (AUC) to the maximum blood concentration (Cₘₐₓ) is 2.16 to 2.18.

Further, the formulation for preventing or treating idiopathic pulmonary fibrosis according to the present invention is characterized in that upon elution under acidic conditions (pH 1.2), the elution rate within 120 minutes after elution is within 10%.

It is well known that pirfenidone has side effects such as vomiting, dermatitis, and loss of appetite. Particularly, it is known that it lowers the medication compliance of patients due to gastrointestinal side effects such as vomiting and loss of appetite.

Because the formulation according to the present invention exhibits unique pharmacokinetic properties as described above, pirfenidone having a high frequency of side effects is not substantially released in a stomach, and is released from a small intestine, thereby substantially alleviating the risk of side effects related to the stomach, or at least reducing it, without affecting the bioavailability of pirfenidone as the active component.

In a specific embodiment, the formulation according to the invention may include a core containing pirfenidone and a pharmaceutically acceptable additive; and two or more mutually different coatings located on the surface of the core.

The core may contain 1 to 1000 mg, preferably 200 to 600 mg of pirfenidone. Specifically, in the formulation according to the present invention, an appropriate content of pirfenidone can be selected within the above range and included in the core so that it can be administered according to the current general administration frequency of pirfenidone (i.e., as the initial dose, 3 times a day, 200 mg of a tablet at a time).

The content of pirfenidone, which is an active component in the formulation according to the present invention, may be appropriately adjusted depending on the form and purpose of use, patient's condition, type and severity of symptoms, and the like, and may be 10 to 99% by weight based on the total weight of the formulation. However, this may be increased or decreased according to the needs of the user, and may be appropriately increased or decreased according to various factors such as diet, nutritional status, and the degree of progression of pathological symptoms, but is not limited to the above range.

Furthermore, the core may contain a pharmaceutically acceptable additive together with pirfenidone as the active component. Examples of such additives may be formulated using a carrier, an excipient, a binder, a disintegrant, a lubricant, a solubilizer, a suspending agent, a preservative and/or a bulking agent, and the like.

Examples of the excipient include lactose hydrate, mannitol, corn starch, microcrystalline cellulose, sucrose, dextrose, and sorbitol, and more preferably lactose hydrate, without being limited thereto. Preferably, the content of the excipient may be 5 to 80% by weight, 10 to 70% by weight, or 15 to 60% by weight of the total weight of the prepared core, without being limited thereto.

The binder includes, preferably, hydroxypropyl cellulose, hydroxypropyl methyl cellulose, methyl cellulose, or polyvinyl pyrrolidone, and more preferably, hydroxypropyl cellulose, without being limited thereto. Preferably, the content of the binder may be 0.5 to 30% by weight, 1 to 25% by weight, or 1.5 to 20% by weight of the total weight of the prepared core, without being limited thereto.

The disintegrant may include, preferably, corn starch, carboxymethyl cellulose, carboxymethyl cellulose calcium, carmellose sodium, low-substituted hydroxypropyl cellulose, croscarmellose sodium, carboxymethyl starch sodium, sodium starch glycolate, cross-linked polyvinylpyrrolidine, and more preferably, croscarmellose sodium, without being limited thereto. Preferably, the content of the disintegrant may be 0.1 to 30% by weight, 0.5 to 25% by weight, or 1 to 20% by weight of the total weight of the tablet, without being limited thereto.

The lubricant may include, preferably, calcium stearate, sodium stearate, magnesium stearate, sodium stearyl fumarate, stearic acid, light anhydrous silicic acid, and more preferably, magnesium stearate, without being limited thereto. Preferably, the content of the lubricant may be 0.1 to 5% by weight based on the total weight of the prepared core, without being limited thereto.

For the purpose of the present invention, the core containing pirfenidone may be preferably formulated in a tablet form, without being limited thereto. Such tablets can be produced according to common tablet production methods (e.g., grinding, mixing, assembling, drying, tableting, coating, etc.). Specifically, in formulating into tablets, the core may be prepared according to a wet granulation method, a dry granulation method, or a direct compression method. In the wet granulation method, the tablet can be produced, for example, through a mixing process in which the active component (main component), diluent, excipient and disintegrant are weighed and mixed with a mixer until they are homogenized, a coalescence process of adding and mixing a binder thereto, a granulation process of sieving and extruding to produce granules, a process of drying the produced granule, an alignment process of aligning the granules again using a small sieve, and a process of adding a lubricant to the aligned granules, post-mixing, and tableting. In addition, in the dry granulation method, the tablet is produced, for example, according to a process of weighing and mixing respective components, a process of slugging or compressing it, a process of producing it into flat tablets or pellets, a process of crushing the tablets or pellets, a slugging process which undergoes a sieving process and a tableting process, or a process of mixing and putting the weighed components into a roller to produce a compressed material, a roller compression method in which the compressed material is crushed, the particles are aligned, lubricated, and then compressed into tablets. Further, the direct compression process refers to a process of mixing the components necessary for producing the tablet and then compressing the components at once. In a preferred embodiment, the core may be formulated in the form of a tablet (core) by a wet granulation process.

The formulation for preventing or treating idiopathic pulmonary fibrosis according to the present invention includes two or more mutually different coatings on the core. The two or more mutually different coatings may be two or more types selected from the group consisting of a coating containing a water-soluble or insoluble polymer and a coating containing an enteric polymer. Preferably, the formulation according to the present invention may include a coating containing a water-soluble or insoluble polymer as a primary coating coated on the surface of the core and a coating containing an enteric polymer as a secondary coating coated on the primary coating. The coating containing the water-soluble or insoluble polymer has the advantage of lubricating the surface of the core and exhibiting effective enteric crystal properties through a constant and stable enteric coating. Furthermore, it is possible to block pirfenidone from the enteric coating made of acidic materials and thus prevent deterioration of stability, and also it is possible to prevent deterioration of the stability of pirfenidone due to the organic solvent used for the enteric coating.

The coating containing the enteric polymer prevents the release of pirfenidone under acidic conditions, and the enteric coating is dissolved from the upper part of the small intestine, allowing the drug to be released.

A coating containing a water-soluble or insoluble polymer and a coating containing an enteric polymer as described above can be specifically combined to make pirfenidone have pharmacokinetic properties unique to the present invention when administered into the body and thus reduce the gastrointestinal side effects caused by the administration of pirfenidone, thereby providing a formulation for oral administration having improved stability of pirfenidone.

In a preferred embodiment, the water-soluble or insoluble polymer may be one type or more selected from the group consisting of ethyl cellulose, hydroxypropylmethyl cellulose, hydroxypropyl cellulose, hydroxyethyl cellulose and polyvinyl alcohol, without being limited thereto. Such a water-soluble or insoluble polymer may be appropriately selected in consideration of the acid resistance of the formulation according to the present invention, prevention of deterioration of pirfenidone stability due to organic solvents, and the relationship between impact resistance and film composition.

Further, the enteric polymer may be a pH-dependent polymer that does not release pirfenidone in an acidic environment such as the stomach. For example, the enteric polymer may be one type of more selected from the group consisting of methacrylic acid copolymer, hydroxypropylmethylcellulose phthalate, hydroxypropylmethylcellulose acetate succinate, cellulose acetate, cellulose acetate phthalate, cellulose acetate succinate, and polyvinyl acetate phthalate. In a preferred embodiment, the enteric polymer may be a methacrylic acid copolymer. The methacrylic acid copolymer is preferable in that it does not impair the stability of pirfenidone during enteric coating.

In a preferable embodiment, the coating containing the enteric polymer may further include at least one plasticizer selected from the group consisting of diethyl phthalate, triethyl phthalate, triethyl citrate, triacetin, tributysebecate and polyethylene glycol; at least one lubricant selected from the group consisting of stearic acid, magnesium stearate and talc; and at least one light-shielding agent selected from the group consisting of titanium oxide and zinc oxide. The plasticizer may be contained in an amount of 0.5 to 15% by weight based on the total weight of the coating containing the enteric polymer, the lubricant may be contained in an amount of 0.1 to 15% by weight based on the total weight of the coating containing the enteric polymer, the light-shielding agent may be contained in an amount of 0.1 to 25% by weight based on the total weight of the coating containing the enteric polymer.

In the formulation according to the present invention, the water-soluble or insoluble polymer is used in a weight ratio of 1:0.005 to 1:0.15, preferably in a weight ratio of 1:0.01 to 1:0.1, and most preferably in a weight ratio of 1:0.02 to 0.05, based on the weight of pirfenidone. <1> At a ratio of 1:0.005 or less, it is difficult to impart sufficient impact resistance to the core and prevent deterioration of stability, and <2> at a ratio of 1:0.15 or more, there is a problem of delaying the elution of the core. In the formulation according to the present invention, the enteric polymer is used in a weight ratio of 1:0.001 to 1:0.3, preferably in a weight ratio of 1:0.02 to 1:0.25, and most preferably in a weight ratio of 1:0.05 to 1:0.2 based on the weight of pirfenidone. <1> At a ratio of 1:0.001 or less, it is difficult to suppress drug release in sufficient acidic conditions and to provide sufficient delay time, and <2> at a ratio of 1:0.3 or more, there is a problem that drug release does not occur or the delay time is excessively exceeded.

The weight ratio of the coating containing the water-soluble or insoluble polymer to the coating containing the enteric polymer may be 1:60 to 1:1.

The formulation for preventing or treating idiopathic pulmonary fibrosis for oral administration according to the present invention can be produced by preparing the core using pirfenidone and a pharmaceutically acceptable additive, and then forming two or more types of coatings on the prepared core using two or more types of polymers. Preferably, the two or more mutually different coatings can be made sequentially, or can be performed by forming through the step of performing a primary coating using a primary coating base containing water-soluble or insoluble polymer and the step of performing a secondary coating base containing an enteric polymer.

In a specific embodiment, the formulation for preventing or treating idiopathic pulmonary fibrosis for oral administration according to the present invention may include the following steps:
a) mixing pirfenidone as an active component with an excipient and a disintegrant;
b) adding a binder to the mixture prepared in step a) to obtain a granule;
c) mixing and tableting the granule prepared in step b) with a lubricant to prepare a core;
d) preparing a primary coated tablet using a primary coating base containing a water-soluble or insoluble polymer for the core prepared in step c);
e) coating the primary coated tablet prepared in step d) with an enteric coating base containing an enteric polymer.

Steps a), b) and c) of the production method are steps performed for producing a core containing pirfenidone and a pharmaceutically acceptable additive thereof. Unless otherwise defined, pirfenidone, as for the matters regarding pharmaceutically acceptable additives, water-soluble or insoluble polymers and enteric polymers, and their contents used in each step above, the matters described for the above formulation can be applied as they are. The step of mixing pirfenidone as an active component with an excipient and a disintegrant in step a) can be performed by selecting various mixing devices such as a known mixer, for example, a fluidized bed mixer, a ribbon mixer, and the like without limitation. Since step b) is a step of adding/mixing a binder to the mixture prepared in step a) to perform coalescence, and sieving and extruding the result to produce a granule, a known coalescence process, a granulation process, and a sizing process can be applied. As a method of preparing the granule in step b), a wet granulation method or a dry granulation method can be applied. In step c), the granule prepared in step b) and a lubricant can be mixed to prepare a core. The core is preferably in the form of a tablet (core).

In order to add the impact resistance or the like of the core prepared in step c), a primary coating tablet (i.e., core+core outer primary coating) is produced using a primary coating base containing a water-soluble or insoluble polymer in step d).

The primary coating base includes at least one water-soluble or insoluble polymer selected from the group consisting of ethyl cellulose, hydroxypropyl cellulose, hydroxypropyl methyl cellulose, hydroxyethyl cellulose and polyvinyl alcohol, without being limited thereto. In an embodiment of the present invention, Opadry® clear (Colorcon, USA), which is hydroxypropylmethylcellulose, was used as the primary coating base.

Step e) is a step in which the primary coated tablet by a primary coating base containing a water-soluble or insoluble polymer is coated with a secondary (enteric) coating base containing methacrylic acid copolymer enteric polymer, which is a pH-dependent polymer, to form an enteric coating on the outermost part of the formulation, finally producing a formulation for preventing or treating idiopathic pulmonary fibrosis according to the present invention. Preferably, the enteric coating base may include at least one plasticizer selected from the group consisting of diethyl phthalate, triethyl phthalate, triethyl citrate, triacetin, tributysebecate and polyethylene glycol; at least one lubricant selected from the group consisting of stearic acid, magnesium stearate and talc; and at least one light-shielding agent selected from the group consisting of titanium oxide and zinc oxide, without being limited thereto. In an embodiment of the present invention, Acryl-Eze® (Colorcon, USA) which is a methacrylic acid copolymer, or hydroxypropylmethylcellulose phthalate HP 50 was used as the primary coating base.

The method of forming the coating layer can be appropriately selected by those skilled in the art among methods capable of forming a film-like coating layer on the surface of the tablet layer using the components described above, and a method such as a fluidized bed coating method and a pan coating method can be applied. Preferably, the pan coating method is used.

The production method according to the present invention can provide a formulation in which a core is prepared by formulating a pharmaceutical composition containing pirfenidone and pharmaceutically acceptable additives, these are designed so that the drug is not released under acidic conditions through two or more coating bases, for example, a primary coating base containing a water-soluble or insoluble polymer and an enteric coating base containing an enteric polymer, so that in actual administration, a drug is not substantially released from the stomach, and can exhibit pharmacokinetic properties that allow the drug to be released only when it reaches the small intestine.

According to an embodiment of the present invention, there is provided a formulation for preventing or treating idiopathic pulmonary fibrosis having improved safety, characterized in that in the stability test under severe conditions (60±2° C.) for 1 month and accelerated conditions (40±2° C., 75±5% RH) for 6 months, related compound A is 0.1% or less, related compound B is 0.1% or less, individual unknown related compounds are 0.05% or less, and total related compounds are 0.3% or less.

### EXAMPLE

Hereinafter, the present invention will be described in more detail with reference to the following examples. However, these examples are for illustrative purposes only and are not intended to limit the scope of the invention in any way.

### Example 1: Preparation I of Pirfenidone 200 mg Enteric-Coated Tablet

Pirfenidone as the active component was mixed with lactose hydrate and croscarmellose sodium. A solution with about 5 (w/w) % concentration of hydroxypropyl cellulose was prepared and sprayed using a fluidized bed granulator to prepare a granule. Croscarmellose sodium was added to the granule and mixed, and then lubricated with magnesium stearate. The final mixture was compressed with a force of 15 kN to obtain a core containing 200 mg of pirfenidone. (core weight 285 mg/tablet). A solution with about 10 (w/w) % concentration of Opadry clear per tablet was prepared and sprayed using a pan coater to coat 6 mg, and then a solution with a concentration of about 10 (w/w) % containing hydroxypropylmethylcellulose phthalate HP55 as an enteric coating base, Methyl citrate as a plasticizer, and talc as a lubricant was prepared and sprayed to perform enteric coating.

The components of the pirfenidone enteric-coated tablet are shown in Table 1 below.

TABLE 1

| Compounding | | Quantity |
|---|---|---|
| purpose | Component nane | (mg) |
| Main component | Pirfenidone (Vegesna Laboratory, | 200 |
| | India) | |
| Excipient | Lactose hydrate (DFE Pharma, | 55 |
| | Germany) | |
| Disintegrant | Croscarmellose sodium (DFE Pharma, | 20 |
| | Germany) | |
| Binder | Hydroxypropyl cellulose (Ashland, | 8 |
| | USA) | |
| Lubricant | Magnesium stearate (NOF Corporation, | 2 |
| | Japan) | |
| Core weight | | 285 |
| Primary | Opadry clear (Colorcon, USA) | 6 |
| coating base | | |
| Primary coated tablet weight | | 291 |
| Enteric | Hydroxypropylmethylcellulose | 26.1 |
| coating base | phthalate HP 55 (Shin-Etsu, Japan) | |
| Plasticizer | Triethylcitrate (Sigma Aldrich, | 2.6 |
| | Germany) | |
| Lubricant | Talc (Nippon Talc, Japan) | 1.3 |

| | |
|---|---|
| Secondary coated tablet weight | 321 |
| Ratio of enteric coating to core weight (w/w %) | 10.5% |

### Example 2: Preparation II of Pirfenidone 200 mg Enteric-Coated Tablet

Pirfenidone as the active component was mixed with lactose hydrate and croscarmellose sodium. A solution with about 5 (w/w) % concentration of hydroxypropyl cellulose was prepared and sprayed using a fluidized bed granulator to prepare a granule. Croscarmellose sodium was added to the granule and mixed, and then lubricated with magnesium stearate. The final mixture was compressed with a force of 15 kN to obtain a core containing 200 mg of pirfenidone. (core weight 285 mg/tablet). A solution with about 10 (w/w) % concentration of Opadry clear per tablet was prepared and sprayed using a pan coater to coat 6 mg, and then a solution with a concentration of about 10 (w/w) % containing hydroxypropylmethylcellulose phthalate HP55 as an enteric coating base, triethyl citrate as a plasticizer, and talc as a lubricant was prepared and sprayed to perform enteric coating.

The components of the pirfenidone enteric-coated tablet are shown in Table 2 below.

TABLE 2

| Compounding | | Quantity |
|---|---|---|
| purpose | Component nane | (mg) |
| Main component | Pirfenidone (Vegesna Laboratory, | 200 |
| | India) | |
| Excipient | Lactose hydrate (DFE Pharma, | 55 |
| | Germany) | |
| Disintegrant | Croscarmellose sodium (DFE Pharma, | 20 |
| | Germany) | |
| Binder | Hydroxypropyl cellulose (Ashland, | 8 |
| | USA) | |
| Lubricant | Magnesium stearate (NOF Corporation, | 2 |
| | Japan) | |
| Core weight | | 285 |
| Primary | Opadry clear (Colorcon, USA) | 6 |
| coating base | | |
| Primary coated tablet weight | | 291 |
| Enteric | Hydroxypropylmethylcellulose | 34.8 |
| coating base | phthalate HP 55 (Shin-Etsu, Japan) | |
| Plasticizer | Triethylcitrate (Sigma Aldrich, | 3.5 |
| | Germany) | |
| Lubricant | Talc (Nippon Talc, Japan) | 1.7 |

| | |
|---|---|
| Secondary coated tablet weight | 331 |
| Ratio of enteric coating to core weight (w/w %) | 14.0% |

### Example 3: Preparation III of Pirfenidone 200 mg Enteric-Coated Tablet

Pirfenidone as the active component was mixed with lactose hydrate and croscarmellose sodium. A solution with about 5 (w/w) % concentration of hydroxypropyl cellulose was prepared and sprayed using a fluidized bed granulator to prepare a granule. Croscarmellose sodium was added to the granule and mixed, and then lubricated with magnesium stearate. The final mixture was compressed with a force of 15 kN to obtain a core containing 200 mg of pirfenidone. (core weight 285 mg/tablet). A solution with about 10 (w/w) % concentration of Opadry clear per tablet was prepared and sprayed using a pan coater to coat 6 mg, and then a solution with a concentration of about 10 (w/w) % containing hydroxypropylmethylcellulose phthalate HP50 as an enteric coating base, triethyl citrate as a plasticizer, and talc as a lubricant was prepared and sprayed to perform enteric coating.

The components of the pirfenidone enteric-coated tablet are shown in Table 3 below.

TABLE 3

| Compounding | | Quantity |
|---|---|---|
| purpose | Component nane | (mg) |
| Main component | Pirfenidone (Vegesna Laboratory, | 200 |
| | India) | |
| Excipient | Lactose hydrate (DFE Pharma, | 55 |
| | Germany) | |
| Disintegrant | Croscarmellose sodium (DFE Pharma, | 20 |
| | Germany) | |
| Binder | Hydroxypropyl cellulose (Ashland, | 8 |
| | USA) | |
| Lubricant | Magnesium stearate (NOF Corporation, | 2 |
| | Japan) | |
| Core weight | | 285 |
| Primary | Opadry clear (Colorcon, USA) | 6 |
| coating base | | |
| Primary coated tablet weight | | 291 |
| Enteric | Hydroxypropylmethylcellulose | 26.1 |
| coating base | phthalate HP 50 (Shin-Etsu, Japan) | |
| Plasticizer | Triethylcitrate (Sigma Aldrich, | 2.6 |
| | Germany) | |
| Lubricant | Talc (Nippon Talc, Japan) | 1.3 |

| | |
|---|---|
| Secondary coated tablet weight | 321 |
| Ratio of enteric coating to core weight (w/w %) | 10.5% |

### Example 4: Preparation IV of Pirfenidone 200 mg Enteric-Coated Tablet

Pirfenidone as the active component was mixed with lactose hydrate and croscarmellose sodium. A solution with about 5 (w/w) % concentration of hydroxypropyl cellulose was prepared and sprayed using a fluidized bed granulator to prepare a granule. Croscarmellose sodium was added to the granule and mixed, and then lubricated with magnesium stearate. The final mixture was compressed with a force of 15 kN to obtain a core containing 200 mg of pirfenidone. (core weight 285 mg/tablet). A solution with about 10 (w/w) % concentration of Opadry clear per tablet was prepared and sprayed using a pan coater to coat 6 mg, and then a solution with a concentration of about 10 (w/w) % containing Eudragit L100-55 as an enteric coating base, triethyl citrate as a plasticizer, and talc as a lubricant was prepared and sprayed to perform enteric coating.

The components of the pirfenidone enteric-coated tablet are shown in Table 4 below.

TABLE 4

| Compounding | | Quantity |
|---|---|---|
| purpose | Component nane | (mg) |
| Main component | Pirfenidone (Vegesna Laboratory, | 200 |
| | India) | |
| Excipient | Lactose hydrate (DFE Pharma, | 55 |
| | Germany) | |
| Disintegrant | Croscarmellose sodium (DFE Pharma, | 20 |
| | Germany) | |
| Binder | Hydroxypropyl cellulose (Ashland, | 8 |
| | USA) | |
| Lubricant | Magnesium stearate (NOF Corporation, | 2 |
| | Japan) | |
| Core weight | | 285 |
| Primary | Opadry clear (Colorcon, USA) | 6 |
| coating base | | |
| Primary coated tablet weight | | 291 |
| Enteric | Eudragit L100-55 (Evonik industries, | 26.1 |
| coating base | Germany) | |
| Plasticizer | Triethylcitrate (Sigma Aldrich, | 2.6 |
| | Germany) | |
| Lubricant | Talc (Nippon Talc, Japan) | 1.3 |

| | |
|---|---|
| Secondary coated tablet weight | 321 |
| Ratio of enteric coating to core weight (w/w %) | 10.5% |

### Example 5: Preparation V of Pirfenidone 200 mg Enteric-Coated Tablet

Pirfenidone as the active component was mixed with lactose hydrate and croscarmellose sodium. A solution with about 5 (w/w) % concentration of hydroxypropyl cellulose was prepared and sprayed using a fluidized bed granulator to prepare a granule. Croscarmellose sodium was added to the granule and mixed, and then lubricated with magnesium stearate. The final mixture was compressed with a force of 15 kN to obtain a core containing 200 mg of pirfenidone. (core weight 285 mg/tablet). A solution with about 10 (w/w) % concentration of Opadry clear per tablet was prepared and sprayed using a pan coater to coat 6 mg, and then a solution with a concentration of about 10 (w/w) % containing hydroxypropylmethylcellulose phthalate HP55 as an enteric coating base, Methyl citrate as a plasticizer, and talc as a lubricant was prepared and sprayed to perform enteric coating.

The components of the pirfenidone enteric-coated tablet are shown in Table 5 below.

TABLE 5

| Compounding | | Quantity |
|---|---|---|
| purpose | Component nane | (mg) |
| Main component | Pirfenidone (Vegesna Laboratory, | 200 |
| | India) | |
| Excipient | Lactose hydrate (DFE Pharma, | 55 |
| | Germany) | |
| Disintegrant | Croscarmellose sodium (DFE Pharma, | 20 |
| | Germany) | |
| Binder | Hydroxypropyl cellulose (Ashland, | 8 |
| | USA) | |
| Lubricant | Magnesium stearate (NOF Corporation, | 2 |
| | Japan) | |
| Core weight | | 285 |
| Primary | Opadry clear (Colorcon, USA) | 6 |
| coating base | | |
| Primary coated tablet weight | | 291 |
| Enteric | hydroxypropylmethylcellulose | 52.2 |
| coating base | phthalate HP55 (Shin-Etsu, Japan) | |
| Plasticizer | Triethylcitrate (Sigma Aldrich, | 5.2 |
| | Germany) | |
| Lubricant | Talc (Nippon Talc, Japan) | 2.6 |

| | |
|---|---|
| Secondary coated tablet weight | 351 |
| Ratio of enteric coating to core weight (w/w %) | 21.1% |

### Example 6: Preparation VI of Pirfenidone 200 mg Enteric-Coated Tablet

Pirfenidone as the active component was mixed with lactose hydrate and croscarmellose sodium. A solution with about 5 (w/w) % concentration of hydroxypropyl cellulose was prepared and sprayed using a fluidized bed granulator to prepare a granule. Croscarmellose sodium was added to the granule and mixed, and then lubricated with magnesium stearate. The final mixture was compressed with a force of 15 kN to obtain a core containing 200 mg of pirfenidone. (core weight 285 mg/tablet). A solution with about 10 (w/w) % concentration of Opadry clear per tablet was prepared and sprayed using a pan coater to coat 6 mg, and then a solution with a concentration of about 10 (w/w) % containing hydroxypropylmethylcellulose phthalate HP55 as an enteric coating base, triethyl citrate as a plasticizer, and talc as a lubricant was prepared and sprayed to perform enteric coating.

The components of the pirfenidone enteric-coated tablet are shown in Table 6 below.

TABLE 6

| Compounding | | Quantity |
|---|---|---|
| purpose | Component nane | (mg) |
| Main component | Pirfenidone (Vegesna Laboratory, | 200 |
| | India) | |
| Excipient | Lactose hydrate (DFE Pharma, | 55 |
| | Germany) | |
| Disintegrant | Croscarmellose sodium (DFE Pharma, | 20 |
| | Germany) | |
| Binder | Hydroxypropyl cellulose (Ashland, | 8 |
| | USA) | |
| Lubricant | Magnesium stearate (NOF Corporation, | 2 |
| | Japan) | |
| Core weight | | 285 |
| Primary | Opadry clear (Colorcon, USA) | 6 |
| coating base | | |
| Primary coated tablet weight | | 291 |
| Enteric | hydroxypropylmethylcellulose | 78.3 |
| coating base | phthalate HP55 (Shin-Etsu, Japan) | |
| Plasticizer | Triethylcitrate (Sigma Aldrich, | 7.8 |
| | Germany) | |
| Lubricant | Talc (Nippon Talc, Japan) | 3.9 |

| | |
|---|---|
| Secondary coated tablet weight | 381 |
| Ratio of enteric coating to core weight (w/w %) | 31.6% |

### Example 7: Preparation VII of Pirfenidone 200 mg Enteric-Coated Tablet

Pirfenidone as the active component was mixed with lactose hydrate and croscarmellose sodium. A solution with about 5 (w/w) % concentration of hydroxypropyl cellulose was prepared and sprayed using a fluidized bed granulator to prepare a granule. Croscarmellose sodium was added to the granule and mixed, and then lubricated with magnesium stearate. The final mixture was compressed with a force of 15 kN to obtain a core containing 200 mg of pirfenidone. (core weight 285 mg/tablet). A solution with about 20 (w/w) % concentration of Opadry clear per tablet was prepared and sprayed using a pan coater to coat 6 mg, and then a solution with a concentration of about 10 (w/w) % of Acryl-Eze white as an enteric coating base to perform enteric coating.

The components of the pirfenidone enteric-coated tablet are shown in Table 7 below.

TABLE 7

| Compounding | | Quantity |
|---|---|---|
| purpose | Component nane | (mg) |
| Main component | Pirfenidone (Vegesna Laboratory, | 200 |
| | India) | |
| Excipient | Lactose hydrate (DFE Pharma, | 55 |
| | Germany) | |
| Disintegrant | Croscarmellose sodium (DFE Pharma, | 20 |
| | Germany) | |
| Binder | Hydroxypropyl cellulose (Ashland, | 8 |
| | USA) | |
| Lubricant | Magnesium stearate (NOF Corporation, | 2 |
| | Japan) | |
| Core weight | | 285 |
| Primary | Opadry clear (Colorcon, USA) | 6 |
| coating base | | |
| Primary coated tablet weight | | 291 |
| Enteric | Acryl-Eze white (Colorcon, USA) | 30 |
| coating base | | |

| | |
|---|---|
| Secondary coated tablet weight | 321 |
| Ratio of enteric coating to core weight (w/w %) | 10.5% |

### Example 8: Preparation VIII of Pirfenidone 200 mg Enteric-Coated Tablet

Pirfenidone as the active component was mixed with lactose hydrate and croscarmellose sodium. A solution with about 5 (w/w) % concentration of hydroxypropyl cellulose was prepared and sprayed using a fluidized bed granulator to prepare a granule. Croscarmellose sodium was added to the granule and mixed, and then lubricated with magnesium stearate. The final mixture was compressed with a force of 15 kN to obtain a core containing 200 mg of pirfenidone. (core weight 285 mg/tablet). A solution with about 10 (w/w) % concentration of Opadry clear per tablet was prepared and sprayed using a pan coater to coat 6 mg, and then a solution with a concentration of about 20 (w/w) % of Acryl-Eze white as an enteric coating base to perform enteric coating.

The components of the pirfenidone enteric-coated tablet are shown in Table 8 below.

TABLE 8

| Compounding | | Quantity |
|---|---|---|
| purpose | Component nane | (mg) |
| Main component | Pirfenidone (Vegesna Laboratory, | 200 |
| | India) | |
| Excipient | Lactose hydrate (DFE Pharma, Germany) | 55 |
| Disintegrant | Croscarmellose sodium (DEE Pharma, | 20 |
| | Germany) | |
| Binder | Hydroxypropyl cellulose (Ashland, | 8 |
| | USA) | |
| Lubricant | Magnesium stearate (NOF Corporation, | 2 |
| | Japan) | |
| Core weight | | 285 |
| Primary | Opadry clear (Colorcon, USA) | 6 |
| coating base | | |
| Primary coated tablet weight | | 291 |
| Enteric | Acryl-Eze white (Colorcon, USA) | 40 |
| coating base | | |

| | |
|---|---|
| Secondary coated tablet weight | 331 |
| Ratio of enteric coating to core weight (w/w %) | 14.0% |

### Comparative Example 1: Preparation of Pirfenidone 200 mg General Coated Tablet

Pirfenidone as the active component was mixed with lactose hydrate and croscarmellose sodium. A solution with about 5 (w/w) % concentration of hydroxypropyl cellulose was prepared and sprayed using a fluidized bed granulator to prepare a granule. Croscarmellose sodium was added to the granule and mixed, and then lubricated with magnesium stearate. The final mixture was compressed with a force of 15 kN to obtain a core containing 200 mg of pirfenidone. (core weight 285 mg/tablet). A solution with about 10 (w/w) % concentration of Opadry clear per tablet was prepared and sprayed using a pan coater to coat 10 mg, thereby obtain a desired pirfenidone general coated tablet. The components of the pirfenidone general coated tablet are shown in Table 9 below.

TABLE 9

| Compounding | | Quantity |
|---|---|---|
| purpose | Component nane | (mg) |
| Main component | Pirfenidone (Vegesna Laboratory, | 200 |
| | India) | |
| Excipient | Lactose hydrate (DFE Pharma, Germany) | 55 |
| Disintegrant | Croscarmellose sodium (DFE Pharma, | 20 |
| | Germany) | |
| Binder | Hydroxypropyl cellulose (Ashland, | 8 |
| | USA) | |
| Lubricant | Magnesium stearate (NOF Corporation, | 2 |
| | Japan) | |
| Core weight | | 285 |
| Coating base | Opadry yellow (Colorcon, USA) | 10 |

| | |
|---|---|
| Coated tablet weight | 295 |

### Test Example 1: Evaluation of Dissolution Test Under Acidic Conditions

An dissolution test was performed on the coated tablets containing pirfenidone prepared according to Examples and Comparative Examples of the present invention using an dissolution solution (pH 1.2, 900 mL) under acidic conditions. The dissolution test was conducted with ERWEKA DISSOLUTION TESTER, and the test solution was collected and analyzed every hour using an Agilent 1260 series HPLC.

<HPLC Analysis Conditions>

Mobile phase: pH 3.0 aqueous solution¹⁾/methanol/acetonitrile=650/130/220 (v/v/v)

Detector: Ultraviolet absorbance spectrometer (measurement wavelength 220 nm)

Flow rate: 1.0 mL/min

Column A column packed with 5 μm octadecylsilyl silica gel in a stainless steel tube with an inner diameter of about 4.6 mm and a length of about 150 mm

Column temperature: constant temperature around 40° C.

Preparation of Standard Solution:

11 mg of the pirfenidone standard was precisely weighed, and put in a 50 mL volumetric flask, and 35 mL of the eluent was added thereto, ultrasonically extracted for 5 minutes, cooled, and then aligned with the marked line with the eluent to make a standard solution.

1) pH 3.0 aqueous solution: a solution in which 0.9 mL of triethylamine was dissolved in 650 mL of water and then adjusted to pH 3.0 with phosphoric acid

<Calculation Formula>

$$Dissolution rate ( \% ) for the indication amount of pirfenidone ( {C}_{12} {H}_{11} {NO}_{2} : 185.2 ) = \frac{{A}_{T}}{{A}_{S}} \times \frac{{W}_{S}}{C} \times P \times 18$$

A_{T}: Peak area of pirfenidone obtained from test solution

A_{S}: Peak area of pirfenidone obtained from standard solution

W_{S}: Collection amount (mg) of standard sample

C: Indication amount of main component per 1 tablet of the drug

P: Purity (%) of standard product of pirfenidone

18: Dilution factor of test solution and standard solution

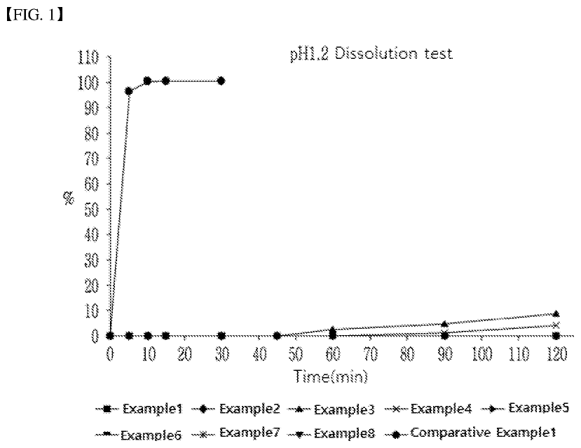

The test results are shown in FIG. 1 and Table 10 below.

TABLE 10

| Time (min) | 5 | 10 | 15 | 30 | 45 | 60 | 90 | 120 |
|---|---|---|---|---|---|---|---|---|
| Example 1 | 0.0 | 0.0 | 0.0 | 0.0 | 0.0 | 0.0 | 0.0 | 0.0 |
| Example 2 | 0.0 | 0.0 | 0.0 | 0.0 | 0.0 | 0.0 | 0.0 | 0.0 |
| Example 3 | 0.0 | 0.0 | 0.0 | 0.0 | 0.0 | 2.4 | 4.6 | 8.7 |
| Example 4 | 0.0 | 0.0 | 0.0 | 0.0 | 0.0 | 0.0 | 1.1 | 3.9 |
| Example 5 | 0.0 | 0.0 | 0.0 | 0.0 | 0.0 | 0.0 | 0.0 | 0.0 |
| Example 6 | 0.0 | 0.0 | 0.0 | 0.0 | 0.0 | 0.0 | 0.0 | 0.0 |
| Example 7 | 0.0 | 0.0 | 0.0 | 0.0 | 0.0 | 0.0 | 0.0 | 0.0 |
| Example 8 | 0.0 | 0.0 | 0.0 | 0.0 | 0.0 | 0.0 | 0.0 | 0.0 |
| Comparative | 96.3 | 100.4 | 100.4 | 100.4 | — | — | — | — |
| Example 1 | | | | | | | | |

As seen in FIG. 1 and Table 10, it can be confirmed that in the case of the pirfenidone formulation according to the present invention, the dissolution rate of pirfenidone at 30 minutes after dissolution under the acidic dissolution condition showed 0% in all of Examples 1 to 4, whereas Comparative Example 1 not including the enteric coating showed an dissolution rate of 90% or more immediately after dissolution, and the entire amount of pirfenidone was released within 30 minutes. According to this result, it can be seen that the formulation of the present invention exhibits an excellent pirfenidone dissolution inhibitory effect under acidic conditions, unlike other formulations.

### Test Example 2: Evaluation of Dissolution Test Under Basic Conditions

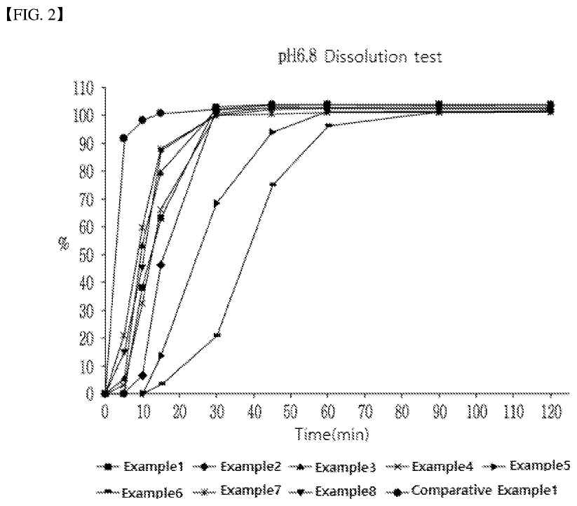

An dissolution test was performed on the coated tablets containing pirfenidone prepared according to Examples and Comparative Examples of the present invention in An dissolution medium (pH6.8, 900 mL) under basic conditions. The dissolution test was conducted with ERWEKAN DISSOLUTION TESTER, and a test solution was collected at each hour using Agilent 1260 series HPLC and analyzed using the analysis conditions and calculation formula of Test Example 1. The test results are shown in FIG. 2 and Table 11 below.

TABLE 11

| Hour (min) | 5 | 10 | 15 | 30 | 45 | 60 | 90 | 120 |
|---|---|---|---|---|---|---|---|---|
| Example 1 | 0.0 | 38.3 | 63.1 | 102.8 | 103.8 | 103.9 | 103.8 | 103.8 |
| Example 2 | 0.0 | 6.7 | 46.3 | 102.1 | 103.6 | 103.8 | 103.6 | 103.6 |
| Example 3 | 5.5 | 53.7 | 79.8 | 102.1 | 102.3 | 102.8 | 102.8 | 102.7 |
| Example 4 | 3.0 | 32.7 | 66.3 | 100.9 | 102.9 | 102.9 | 102.8 | 102.8 |
| Example 5 | 0.0 | 0.0 | 13.8 | 68.7 | 94.1 | 101.3 | 101.4 | 101.8 |
| Example 6 | 0.0 | 0.0 | 3.3 | 20.8 | 74.9 | 96.3 | 101.0 | 101.6 |
| Example 7 | 20.9 | 59.8 | 88.0 | 100.1 | 100.5 | 101.0 | 101.0 | 101.1 |
| Example 8 | 14.7 | 45.3 | 87.1 | 99.9 | 102.1 | 102.3 | 102.2 | 102.2 |
| Comparative | 91.8 | 98.3 | 100.6 | 102.1 | — | — | — | — |
| Example 1 | | | | | | | | |

As seen in FIG. 2 and Table 11, it can be confirmed that in the case of the pirfenidone formulation according to the present invention, the dissolution of pirfenidone started within 10 minutes after dissolution under basic dissolution conditions, and most pirfenidone dissolution was completed within 30 minutes to 2 hours after elution.

### Test Example 3: Pharmacokinetic Analysis Test Using Beagle Dogs

A pharmacokinetic analysis test was performed on coated tablets containing pirfenidone prepared according to Examples and Comparative Examples of the present invention using beagle dogs. The group composition consisted of 10 male beagle dogs, 5 per group, and divided into Example 1 group and Comparative example 1 group. It was washed-out for at least 7 days after oral administration and proceeded with a crossover test. One dog per breeding box made of stainless steel was bred, and 300 g of feed and groundwater treated with reverse osmosis were supplied once a day through an automatic water supply device. Fasting was performed for 16 hours before administration, and drinking water was allowed to be consumed freely. At the time of drug administration, the administrator lightly opened the mouth of the beagle dog with one hand and then put the tablet up to the tongue root with the other hand. At this time, about 10 mL of drinking water was administered for smooth administration. The group composition is shown in Table 12 below.

TABLE 12

| | | | Number of animal |
|---|---|---|---|
| -th time | Test group | | individuals |
| 1 | G1 | Comparative | 5(1M01~1M05) |
| | | Example1 | |
| | G2 | Example 1 | 5(2M01~2M05) |
| 2 | G3 | Example 1 | 5(3M01~3M05) |
| | G4 | Comparative | 5(4M01~4M05) |
| | | Example1 | |

After administration to all individuals of each group, about 2 mL of blood was collected from the forelimb vein using a disposable syringe. The collected blood was immediately put in a blood sample container containing an anticoagulant, and the blood and anticoagulant were mixed using a Roll-mixer (5 Roll Mixer, HYUNIL LAB-MATE). Plasma was separated by centrifugation (Cenrtifuge5810R, Effendorf) at 3,000 rpm for 15 minutes within 30 minutes of collecting blood. The collected blood was centrifuged (Cenrtifuge5810R, Effendorf) for 15 minutes at 3,000 rpm within 30 minutes after collection to separate plasma Each of the separated plasma was put by 400 μl into a prepared tube and stored in a cryogenic refrigerator. Blood sampling times were 0, 0.25, 0.5, 0.75, 1, 2, 3, 4, 6, 8, 10, 12, and 24 hours, and a total of 12 points were collected except for blanks.

<HPLC Analysis Conditions>

Mobile phase: 10 mM ammonium formate (0.1% formic acid): Methanol=4:6 (v/v)

Detector: 4000 Qtrap MRM mode

Flow rate: 0.25 mL/min

Column: A column packed with 3 μm of octadecylsilyl silica gel in a stainless steel tube with an inner diameter of about 2.1 mm and a length of about 50 mm

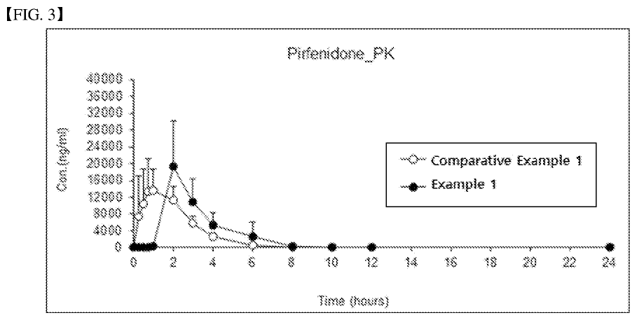

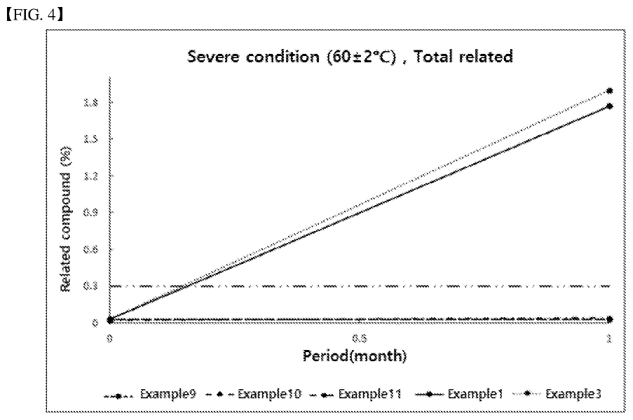

In pharmacokinetic analysis test, the plasma peak concentration (Cₘₐₓ), plasma peak concentration time (Tₘₐₓ), and area under the drug-time curve (AUC) were measured, and the presence or absence of vomiting was observed as a clinical symptom. AUC, Cₘₐₓ and Tₘₐₓ were shown in FIG. 3 and Table 13 below, and the presence or absence of vomiting in individuals after administration and the number of individuals who have vomited are shown in Table 14 below.

TABLE 13

| | Comparative Example 1 | Example 1 |
|---|---|---|
| AUC(₀₋₂₄)(ng · hr/mL) | 37157.49 ± 7331.39 | 39743.33 ± 11555.56 |
| Cₘₐₓ | 17136.40 ± 4959.75 | 18365.58 ± 6210.55 |
| Tₘₐₓ | 1.08 ± 0.755 | 2.29 ± 1.90 |
| T_{1/2} | 0.82 ± 0.08 | 0.86 ± 0.09 |
| AUC/Cₘₐₓ ratio | 2.17 ± 1.48 | 2.16 ± 1.86 |

* Excludes individuals who have vomited.

TABLE 14

| | | | Number of animal | Vomiting |
|---|---|---|---|---|
| -th time | Test group | | individuals | individuals |
| 1 | G1 | Comparative | 5(1M01~1M05) | 2(1M02, 1M03) |
| | | Example 1 | | |
| | G2 | Example 1 | 5(2M01~2M05) | None |
| 2 | G3 | Example 1 | 5(3M01~3M05) | None |
| | G4 | Comparative | 5(4M01~4M05) | 1(4M02) |
| | | Example 1 | | |

As seen in Tables 13 and 14, it can be confirmed that when Comparative Example 1 is administered, the maximum blood concentration (Cₘₐₓ) is reached within an early time after administration, whereas when the formulation of Example 1 according to the present invention is administered, the time to reach the maximum plasma concentration (Tₘₐₓ) is 2.29±1.90 hours after administration, which indicates that a sufficient amount of pirfenidone is released when it reaches the small intestine without being released during passage through the stomach.

### Example 9: Preparation I of Pirfenidone 200 mg Enteric-Coated Tablet

Pirfenidone as the active component was mixed with lactose hydrate and croscarmellose sodium. A solution with about 5 (w/w) % concentration of hydroxypropyl cellulose was prepared and sprayed using a fluidized bed granulator to prepare a granule. Croscarmellose sodium was added to the granule and mixed, and then lubricated with magnesium stearate. The final mixture was compressed with a force of 15 kN to obtain a core containing 200 mg of pirfenidone. (core weight 285 mg/tablet). A solution with about 10 (w/w) % concentration of Opadry clear per tablet was prepared and sprayed using a pan coater to coat 6 mg, and then a solution with a concentration of about 20 (w/w) % of Acryl-Eze white as an enteric coating base to perform enteric coating.

The components of the pirfenidone enteric-coated tablet are shown in Table 15 below.

TABLE 15

| Compounding | | Quantity |
|---|---|---|
| purpose | Component nane | (mg) |
| Main component | Pirfenidone (Vegesna Laboratory, | 200 |
| | India) | |
| Excipient | Lactose hydrate (DFE Pharma, Germany) | 55 |
| Disintegrant | Croscarmellose sodium (DFE Pharma, | 20 |
| | Germany) | |
| Binder | Hydroxypropyl cellulose (Ashland, | 8 |
| | USA) | |
| Lubricant | Magnesium stearate (NOF Corporation, | 2 |
| | Japan) | |
| Core weight | | 285 |
| Primary coating | Opadry clear (Colorcon, USA) | 6 |
| base | | |
| Primary coated tablet weight | | 291 |
| Enteric coating | Acryl-Eze white (Colorcon, USA) | 30 |
| base | | |

| | |
|---|---|
| Secondary coated tablet weight | 321 |
| Ratio of enteric coating to core weight (w/w %) | 10.5% |

### Example 10: Preparation II of Pirfenidone 200 mg Enteric-Coated Tablet

Pirfenidone as the active component was mixed with lactose hydrate and croscarmellose sodium. A solution with about 5 (w/w) % concentration of hydroxypropyl cellulose was prepared and sprayed using a fluidized bed granulator to prepare a granule. Croscarmellose sodium was added to the granule and mixed, and then lubricated with magnesium stearate. The final mixture was compressed with a force of 15 kN to obtain a core containing 200 mg of pirfenidone. (core weight 285 mg/tablet). A solution with about 10 (w/w) % concentration of Opadry clear per tablet was prepared and sprayed using a pan coater to coat 6 mg, and then a solution with a concentration of about 20 (w/w) % of Acryl-Eze white as an enteric coating base to perform enteric coating.

The components of the pirfenidone enteric-coated tablet are shown in Table 16 below.

TABLE 16

| Compounding | | Quantity |
|---|---|---|
| purpose | Component nane | (mg) |
| Main component | Pirfenidone (Vegesna Laboratory, | 200 |
| | India) | |
| Excipient | Lactose hydrate (DFE Pharma, Germany) | 55 |
| Disintegrant | Croscarmellose sodium (DFE Pharma, | 20 |
| | Germany) | |
| Binder | Hydroxypropyl cellulose (Ashland, | 8 |
| | USA) | |
| Lubricant | Magnesium stearate (NOF Corporation, | 2 |
| | Japan) | |
| Core weight | | 285 |
| Primary coating | Opadry clear (Colorcon, USA) | 6 |
| base | | |
| Primary coated tablet weight | | 291 |
| Enteric coating | Acryl-Eze white (Colorcon, USA) | 40 |
| base | | |

| | |
|---|---|
| Secondary coated tablet weight | 331 |
| Ratio of enteric coating to core weight (w/w %) | 14.0% |

### Example 11: Preparation III of Pirfenidone 200 mg Enteric-Coated Tablet

Pirfenidone as the active component was mixed with lactose hydrate and croscarmellose sodium. A solution with about 5 (w/w) % concentration of hydroxypropyl cellulose was prepared and sprayed using a fluidized bed granulator to prepare a granule. Croscarmellose sodium was added to the granule and mixed, and then lubricated with magnesium stearate. The final mixture was compressed with a force of 15 kN to obtain a core containing 200 mg of pirfenidone. (core weight 285 mg/tablet). A solution with about 10 (w/w) % concentration of Opadry clear per tablet was prepared and sprayed using a pan coater to coat 6 mg, and then a solution with a concentration of about 10 (w/w) % containing Eudragit L100-55 as an enteric coating base, triethyl citrate as a plasticizer, and talc as a lubricant was prepared and sprayed to perform enteric coating.

The components of the pirfenidone enteric-coated tablet are shown in Table 17 below.

TABLE 17

| Compounding | | Quantity |
|---|---|---|
| purpose | Component nane | (mg) |
| Main component | Pirfenidone (Vegesna Laboratory, | 200 |
| | India) | |
| Excipient | Lactose hydrate (DFE Pharma, | 55 |
| | Germany) | |
| Disintegrant | Croscarmellose sodium (DFE Pharma, | 20 |
| | Germany) | |
| Binder | Hydroxypropyl cellulose (Ashland, | 8 |
| | USA) | |
| Lubricant | Magnesium stearate (NOF Corporation, | 2 |
| | Japan) | |
| Core weight | | 285 |
| Primary coating | Opadry clear (Colorcon, USA) | 6 |
| base | | |
| Primary coated tablet weight | | 291 |
| Enteric coating | Eudragit L100-55 (Evonik industries, | 26.1 |
| base | Germany) | |
| Plasticizer | Triethylcitrate (Sigma Aldrich, | 2.6 |
| | Germany) | |
| Lubricant | Talc (Nippon Talc, Japan) | 1.3 |

| | |
|---|---|
| Secondary coated tablet weight | 321 |
| Ratio of enteric coating to core weight (w/w %) | 10.5% |

### Test Example 4: Related Compound Test

The coated tablets containing pirfenidone prepared according to Examples to the present invention were stored under severe conditions (60±2° C.) for 1 month and under accelerated conditions (40±2° C., 75±5% RH) for 6 months, and then the sample was collected and analyzed according to the following conditions.

<HPLC Analysis Conditions>

Mobile phase A: After dissolving 0.9 mL of triethylamine in 650 mL of water, the pH is adjusted to 3.0 with phosphoric acid.
Mobile phase B: methanol and acetonitrile are mixed in a volume ratio of 130:220.
Diluent: mobile phase A and mobile phase B are mixed in a ratio of 650:350.
Standard solution: 5 mg of related compound A standard and 5 mg of related compound B standard are weighed, put in a 50 mL volumetric flask, and a diluent is added and dissolved, and aligned with the marked line to make the related compound standard stock solution. 1.0 mL of the test solution is taken, put in a 100 mL volumetric flask, and the diluent is added to align the marked line. 5.0 mL of this solution and 1.0 mL of the related compound standard stock solution are taken and put in a 100 mL volumetric flask. The diluent is added to make exactly 100 mL, which is as a standard solution (related compounds A and B: 1.0 μg/mL, pirfenidone: 0.5 μg/mL).
Test solution: About 20 tablets are taken and precisely weighed to make powder. The amount corresponding to about 100 mg of pirfenidone is accurately weighed and put in a 100 mL volumetric flask, and 70 mL of the dilute is added, and ultrasonically extracted for 5 minutes, and then cooled to a room temperature. Then, the diluent is added to align the marked line. This solution is filtered through a 0.45 an membrane filter to make a test solution (1.0 mg/ml). 20 μL each of the standard solution and the test solution are tested according to the liquid chromatography method under the following conditions.
Operating conditions

Detector: Ultraviolet Absorption Spectrometer

Related compounds A, B (measurement wavelength: 310 nm, Reference: 380 nm)

Unknown related compounds (measurement wavelength: 220 nm, Reference: 380 nm)

Column: packed with a 5 μm octadecylsilyl silica gel for liquid chromatography in a stainless steel tube with an inner diameter of about 4.6 mm and a length of about 250 mm

Column temperature: constant temperature around 40° C.

Mobile phase: Mobile phases A and B are used and controlled stepwise or by concentration gradient as shown in Table 18 below.

TABLE 18

| Time (min) | Mobile phase A(%) | Mobile phase B(%) |
|---|---|---|
| 0 | 98 | 2 |
| 3 | 98 | 2 |
| 40 | 30 | 70 |
| 45 | 30 | 70 |
| 46 | 98 | 2 |
| 50 | 98 | 2 |

Flow rate: 1.0 mL/min

Test time: 50 minutes
System compatibility: When 20 μL of standard solution is repeatedly tested 6 times under the above conditions, the relative standard deviation (% RSD) of the peak areas of related compounds A, related compounds B and pirfenidone should be 2.0% or less. When 20 μL of standard solution is tested under the above conditions, the degree of separation of related compound A and related compound B should be at least 10.
Calculation formula for related substances

$$Amount ( \% ) of Related Compound A and B = \frac{{A}_{T}}{{A}_{S}} \times \frac{{W}_{S}}{{W}_{T}} \times \frac{{W}_{S}}{C} \times 0.02 \times P$$

A_{T}: Peak area of related compounds obtained from 310 nm of test solution

A_{S}: Peak area of related compounds obtained from 310 nm of standard solution

W_{S}: Collection amount (mg) of standard sample

W_{T}: Collection amount (mg) of test sample

M_{N}: Average mass (mg) of 1 tablet of the drug

C: Indication amount (200 mg) of main component per 1 tablet of the drug

0.02: Dilution factor of standard solution and test solution

P: Purity (%) of standard product of related compounds

$$Amount ( \% ) of individual unknown related compounds = \frac{{A}_{T}}{{A}_{S}} \times \frac{{C}_{S}}{{C}_{T}} \times 100$$

A_{T}: Peak area of individual unknown related compounds obtained from 220 nm of test solution^{node1)}

A_{S}: Peak area of pirfenidone obtained from 220 nm of standard solution

C_{S}: Concentration (mg/ml) of pirfenidone among standard solution

C_{T}: Concentration (mg/ml) of pirfenidone among test solution

Total Related Compound (%)=Total Sum of Amount (%) of Related Compound A+Amount (%) of Related Compound B+Amount (%) of Individual Unknown Related Compounds

Note 1) exclude the peaks of related substances A and B at 220 nm wavelength

In addition, the relative retention time and criteria for respective related compounds are as follows.

TABLE 19

| | Relative retention time(RRT) | Criteria |
|---|---|---|
| Related compound A | about 0.13 | 0.1% or less |
| Related compound B | about 0.43 | 0.1% or less |
| Individual unknown | — | 0.05% or less |
| related compound | | |
| Total related compound | — | 0.3% or less |

Test results for the related compounds are shown in Tables 20 to 24 below.

TABLE 20

| | Exam- | Exam- | Exam- | Exam- | Exam- |
|---|---|---|---|---|---|
| | ple 9 | ple 10 | ple 11 | ple 1 | ple 3 |
| Related compound A | ND | ND | ND | ND | ND |
| Related compound B | ND | ND | ND | ND | ND |
| Individual unknown | 0.03 | 0.03 | 0.02 | 0.03 | 0.03 |
| related compound | | | | | |
| Total related | 0.03 | 0.03 | 0.02 | 0.03 | 0.03 |
| compound | | | | | |

TABLE 21

| | Exam- | Exam- | Exam- | Exam- | Exam- |
|---|---|---|---|---|---|
| | ple 9 | ple 10 | ple 11 | ple 1 | ple 3 |
| Related compound A | ND | ND | ND | ND | ND |
| Related compound B | ND | ND | ND | ND | ND |
| Individual known | 0.03 | 0.03 | 0.04 | 1.44 | 1.61 |
| related compound | | | | | |
| Total related | 0.03 | 0.03 | 0.04 | 1.77 | 1.90 |
| compound | | | | | |

TABLE 22

| | Exam- | Exam- | Exam- | Exam- | Exam- |
|---|---|---|---|---|---|
| | ple 9 | ple 10 | ple 11 | ple 1 | ple 3 |
| Related compound A | ND | ND | ND | ND | ND |
| Related compound B | ND | ND | ND | ND | ND |
| Individual unknown | 0.03 | 0.03 | 0.03 | 0.33 | 0.39 |
| related compound | | | | | |
| Total related | 0.03 | 0.03 | 0.03 | 0.55 | 0.62 |
| compound | | | | | |

TABLE 23

| | Exam- | Exam- | Exam- | Exam- | Exam- |
|---|---|---|---|---|---|
| | ple 9 | ple 10 | ple 11 | ple 1 | ple 3 |
| Related compound A | ND | ND | ND | ND | ND |
| Related compound B | ND | ND | ND | ND | ND |
| Individual unknown | 0.03 | 0.03 | 0.04 | 0.70 | 0.77 |
| related compound | | | | | |
| Total related | 0.03 | 0.03 | 0.04 | 1.03 | 1.06 |
| compound | | | | | |

TABLE 24

| | Exam- | Exam- | Exam- | Exam- | Exam- |
|---|---|---|---|---|---|
| | ple 9 | ple 10 | ple 11 | ple 1 | ple 3 |
| Related compound A | ND | ND | ND | ND | ND |
| Related compound B | ND | ND | ND | ND | ND |
| Individual unknown | 0.03 | 0.03 | 0.04 | 1.11 | 1.16 |
| related compound | | | | | |
| Total related | 0.03 | 0.03 | 0.04 | 1.54 | 1.49 |
| compound | | | | | |

From the results of Tables 20 to 24, it can be confirmed that in the case of the formulations according to Examples 9 to 11 using the methacrylic acid copolymer as the enteric coating base, a small amount of related substances was detected as compared with the formulations of Examples 1 and 3 prepared using hydroxypropylmethylcellulose phthalate HP 55 or hydroxypropylmethylcellulose phthalate HP 50 as an enteric coating base rather than methacrylic acid copolymer.

1. A formulation for preventing or treating idiopathic pulmonary fibrosis having improved safety, comprising pirfenidone as an active component, wherein the time to reach the maximum blood concentration (Tₘₐₓ) at the time of administration is 1.2 hours or more after administration.

2. The formulation for preventing or treating idiopathic pulmonary fibrosis having improved safety according to claim 1, comprising a core containing pirfenidone and a pharmaceutically acceptable additive; and two or more mutually different coatings located outside the core, wherein the two or more mutually different coatings are composed of a coating containing a water-soluble or insoluble polymer and a coating containing an enteric polymer.

3. The formulation for preventing or treating idiopathic pulmonary fibrosis having improved safety according to claim 1, wherein the time to reach the maximum blood concentration is 2 to 4 hours after administration.

4. The formulation for preventing or treating idiopathic pulmonary fibrosis having improved safety according to claim 1, wherein the cumulative blood concentration within 1 hour after administration is 0 to 5% of the maximum blood concentration.

5. The formulation for preventing or treating idiopathic pulmonary fibrosis having improved safety according to claim 1, wherein the pirfenidone is contained in an amount of 10 to 99% by weight based on the total weight of the formulation.

6. The formulation for preventing or treating idiopathic pulmonary fibrosis having improved safety according to claim 1, wherein the pirfenidone is contained in an amount of 200 to 600 mg.

7. The formulation for preventing or treating idiopathic pulmonary fibrosis having improved safety according to claim 2, wherein the weight ratio of the coating containing a water-soluble or insoluble polymer and the coating containing an enteric polymer is 1:60 to 1:1.

8. The formulation for preventing or treating idiopathic pulmonary fibrosis having improved safety according to claim 2, wherein the water-soluble or insoluble polymer is at least one selected from the group consisting of ethyl cellulose, hydroxypropylmethyl cellulose, hydroxypropyl cellulose, hydroxyethyl cellulose, polyvinyl alcohol hydroxypropyl methyl cellulose, and polyvinyl alcohol.

9. The formulation for preventing or treating idiopathic pulmonary fibrosis having improved safety according to claim 2, wherein the enteric polymer comprises at least one enteric polymer selected from the group consisting of methacrylic acid copolymer, hydroxypropylmethylcellulose acetate succinate, cellulose acetate, cellulose acetate phthalate, cellulose acetate succinate, and polyvinyl acetate phthalate.

10. The formulation for preventing or treating idiopathic pulmonary fibrosis having improved safety according to claim 9, wherein the enteric polymer comprises a methacrylic acid copolymer.

11. The formulation for preventing or treating idiopathic pulmonary fibrosis having improved safety according to claim 2, wherein the coating containing an enteric polymer further comprises at least one plasticizer selected from the group consisting of diethyl phthalate, triethyl phthalate, triethyl citrate, triacetin, tributyl sebecate and polyethylene glycol.

12. A method for producing the formulation for preventing or treating idiopathic pulmonary fibrosis having improved safety as set forth in claim 1, the method comprising the steps of:
a) mixing pirfenidone as an active component with an excipient and a disintegrant;
b) adding a binder to the mixture prepared in step a) to obtain a granule;
c) mixing and tableting the granule prepared in step b) with a lubricant to prepare a core;
d) preparing a primary coated tablet using a primary coating base containing a water-soluble or insoluble polymer for the core prepared in step c);
e) coating the primary coated tablet prepared in step d) with an enteric coating base containing an enteric polymer.

13. The method for producing the formulation for preventing or treating idiopathic pulmonary fibrosis having improved safety according to claim 12, wherein the primary coating base comprises at least one water-soluble or insoluble polymer selected from the group consisting of ethyl cellulose, hydroxypropyl methyl cellulose, hydroxypropyl cellulose, hydroxyethyl cellulose, polyvinyl alcohol hydroxypropyl methyl cellulose and polyvinyl alcohol.

14. The method for producing the formulation for preventing or treating idiopathic pulmonary fibrosis having improved safety according to claim 12, wherein the enteric coating base comprises at least one enteric polymer selected from the group consisting of methacrylic acid copolymer, hydroxypropylmethylcellulose acetate succinate, cellulose acetate, cellulose acetate phthalate, cellulose acetate succinate, and polyvinyl acetate phthalate.

15. The method for producing the formulation for preventing or treating idiopathic pulmonary fibrosis having improved safety according to claim 14, wherein the enteric coating base comprises a methacrylic acid copolymer.

16. The method for producing the formulation for preventing or treating idiopathic pulmonary fibrosis having improved safety according to claim 12, wherein the enteric coating base comprises at least one plasticizer selected from the group consisting of diethyl phthalate, triethyl phthalate, triethyl citrate, triacetin, tributysebecate and polyethylene glycol; at least one lubricant selected from the group consisting of stearic acid, magnesium stearate and talc; and at least one light-shielding agent selected from the group consisting of titanium oxide and zinc oxide.
